(19) United States
(12) Patent Application Publication
(10) Pub. No.: US 2026/0034214 A1
(43) Pub. Date: Feb. 5, 2026

(54) TREATMENT OF MHC-I NEGATIVE TUMORS WITH NK AND T CELLS

(71) Applicant: ImmunityBio, Inc., San Diego, CA (US)

(72) Inventor: Patrick Soon-Shiong, Culver City, CA (US)

(73) Assignee: ImmunityBio, Inc., San Diego, CA (US)

(21) Appl. No.: 18/997,483

(22) PCT Filed: Jul. 24, 2023
(86) PCT No.: PCT/US2023/028485

§ 371 Date: Jan. 21, 2025

Related U.S. Application Data
(60) Provisional application No. 63/391,912, filed on Jul. 25, 2022.

(51) Int. Cl. A61K 40/11; A61K 31/20; A61K 31/502; A61K 31/573; A61K 31/7068; A61K 31/7072; A61K 31/7105; A61K 35/17; A61K 38/20; A61K 38/21; A61K 40/15; A61K 40/31; A61K 40/32; A61K 40/35; A61K 40/42; A61K 45/06; A61P 35/00; C07K 16/00; G01N 33/68

(52) U.S. Cl. CPC A61K 40/11; A61K 31/20; A61K 31/502; A61K 31/573; A61K 31/7068; A61K 31/7072; A61K 31/7105; A61K 35/17; A61K 38/2086; A61K 38/217; A61K 40/15; A61K 40/31; A61K 40/32; A61K 40/35; A61K 40/4235; A61K 45/06; A61P 35/00; C07K 16/00; G01N 33/6893

(57) ABSTRACT

Methods and compositions are provided herein whereby a patient with cancer is administered a treatment comprising NK cells in order to induce expression of MHC-I in a tumor, and wherein subsequent treatment with T cells effectively targets tumor associated antigens and neoepitopes presented by newly expressed MHC-I in said tumor cells.

This application claims the benefit of priority to U.S. Provisional Application No. 63/391,912 filed on Jul. 25, 2022. This and all other extrinsic references referenced herein are incorporated by reference in their entirety.

### FIELD OF THE INVENTION

The field of the invention is combination immunotherapy technologies.

### BACKGROUND

Major Histocompatability Complex (MHC) proteins are found on the surface of all nucleated cells. They are responsible for presenting intracellularly digested peptides on the cell surface whereby they are screened for non-self peptide sequences by T cells. Peptide sequences from non-self sources such as cancer and infectious pathogens are presented on the cell surface bound to MHC, whereby T cells bind to such occupied MHC proteins and initiate various immune defense programs including cytokine production and direct cytotoxicity. Intracellular proteins are presented on the cell surface by MHC-I, while extracellular proteins are ingested by phagocytosis and processed for presentation by MHC-II. MHC-I is expressed on the surface of all nucleated cells and is responsible for presentation of mutated (non-self) peptides of cellular origin.

Alternatively, natural killer (NK) cell mediated cytotoxicity is inhibited by the presence of MHC-I expressed on the surface of target cells. As infectious agents have mechanisms to reduce expression of MHC in infected cells, thereby mitigating the host's ability to mount a T cell mediated immunological defense, an NK cell mediated defense is enabled by reduced MHC expression in infected cells.

Loss of MHC proteins has been described in cancer cells, whereby T cell activity is diminished. Typically, loss of MHC is associated with lower amounts of tumor infiltrating lymphocytes. Thus, a patient is unable to mount an effective immune response directed to non-self peptides in cancerous or infected tissues. MHC-I expression is inducible with well characterized induction by various agents, prominent among which is interferon-g. Induction of MHC-I followed by T-cell therapy may be an effective therapeutic approach.

Thus, there is still a need for effective therapies which enable and increase a host immune response to cancerous tissue.

### SUMMARY

The inventive subject matter provides compositions, methods, and kits by which an immune response to a cancer or infectious disease may be enabled or enhanced.

In an embodiment, the inventive subject matter comprises a method of treatment for cancer, the method comprising determining the MHC-I expression levels in a tumor. Upon determination that MHC-I levels are low relative to at least a control non-cancerous tissue, the patient is administered a plurality of NK cells, wherein the NK cells comprise at least one of an aNK cell, a haNK cell, a t-haNK cell, or a primary ceNK or memory-like ceNK (m-ceNK) cell. The NK cell may be administered intravenously or intratumorally. After a period of time, the patient is administered a plurality of T cells. The T cells may be autologous or heterologous. The T cell may further be genetically engineered to express a targeting agent, wherein the agent comprises a CAR or a TCR. In a non-limiting example, the T cells may be administered to the patient up to 1 hour after administering the NK cells, or up to 2 hours after administering the NK cells, or up to 5 hours after administering the NK cells, or up to 10 hours after administering the NK cells, or up to 1 day after administering the NK cells, or up to 5 days after administering the NK cells, or up to 1 week after administering the NK cells, or up to 2 weeks after administering the NK cells, or up to 1 month after administering the NK cells, or up to 2 months after administering the NK cells, or up to 6 months after administering the NK cells, or up to 1 year after administering the NK cells, or up to 2 years after administering the NK cells.

In an embodiment, the inventive subject matter comprises a method of treatment for infectious disease, the method comprising determining the MHC-I expression levels in an affected tissue. Upon determination that MHC-I levels are low relative to control healthy normal tissue, the patient is administered a plurality of NK cells, wherein the NK cells comprise at least one of an aNK cell, a haNK cell, a t-haNK cell, or a primary ceNK or memory-like ceNK (m-ceNK) cell. The NK cell may be administered intravenously or intratumorally. After a period of time, the patient is administered a plurality of T cells. The T cells may be autologous or heterologous. The T cell may further be genetically engineered to express a targeting agent, wherein the agent comprises a CAR or a TCR.

In an embodiment, the inventive subject matter comprises an immunotherapeutic composition, the composition comprising a plurality of NK cells, wherein the NK cells comprise at least one of an aNK cell, a haNK cell, a t-haNK cell, a ceNK cell, or an m-ceNK cell. The NK cell may be formulated as a plurality of whole cells for intravenous administration, or it may be formulated as a cell lysate for intratumoral administration. The composition further comprises a plurality of T cells, wherein the T cells are formulated for intravenous administration. The T cells may be autologous or heterologous. The T cell may further be genetically engineered to express a targeting agent, wherein the agent comprises a CAR or a TCR.

In an embodiment, the inventive subject matter comprises the use of an immunotherapeutic composition in the treatment of cancer or infectious disease, the use comprising determining the MHC-I expression levels in an affected tissue, and the composition comprising a plurality of NK cells, wherein the NK cells comprise at least one of an aNK cell, a haNK cell, a t-haNK cell, a ceNK cell, or an m-ceNK cell. The NK cell may be formulated as a plurality of whole cells for intravenous administration, or it may be formulated as a cell lysate for intratumoral administration. The composition further comprises a plurality of T cells, wherein the T cells are formulated for intravenous administration. The T cells may be autologous or heterologous. The T cell may further be genetically engineered to express a targeting agent, wherein the agent comprises a CAR or a TCR.

The method of treatment for cancer or infectious disease may further comprise administration of a secondary agent to be administered concurrently with the NK cells. The agent may comprise at least one therapeutic agent selected from the group consisting of Gemcitabine, IFNg, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, a SMAC mimetic, IL-15, N-803, an IL-15 derivative, and an autophagy inhibitor.

The immunotherapeutic composition may further comprise a secondary agent to be administered concurrently with the NK cells. The agent may comprise at least one therapeutic agent selected from the group consisting of Gemcitabine, IFNg, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, a SMAC mimetic, IL-15, N-803, an IL-15 derivative, and an autophagy inhibitor.

The immunotherapeutic composition for use in the treatment of cancer or infectious disease may further comprise a secondary agent to be administered concurrently with the NK cells. The agent may comprise at least one therapeutic agent selected from the group consisting of Gemcitabine, IFNg, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, a SMAC mimetic, IL-15, N-803, an IL-15 derivative, and an autophagy inhibitor.

In an embodiment, the inventive subject matter comprises a method of induction of MHC-I in a cancer, the method comprising determining MHC-I expression levels in a tumor. Upon determination that MHC-I levels are low relative to control non-cancerous tissue, the patient is administered a plurality of NK cells, wherein the NK cells comprise at least one of an aNK cell, a haNK cell, a t-haNK cell, a ceNK cell, or an m-ceNK cell. The NK cell may be administered intravenously or intratumorally.

In an embodiment, the inventive subject matter comprises a method of increasing levels of IFNg in a cancer, the method comprising determining IFNg expression levels in a tumor. Upon determination that IFNg levels are low relative to control non-cancerous tissue, the patient is administered a plurality of NK cells, wherein the NK cells comprise at least one of an aNK cell, a haNK cell, a t-haNK cell, a ceNK cell, or an m-ceNK cell. The NK cell may be administered intravenously or intratumorally.

In an embodiment, the inventive subject matter comprises a method for determining the MHC-I inducibility of a tumor. In an aspect of the invention, a patient biopsy sample is enzymatically and/or mechanically dissociated ex vivo and individual aliquots are seeded into individual wells of a multiwall microplate. The samples are then exposed to agents known to induce MHC-I, wherein the agents comprise Gemcitabine, IFNg, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, a SMAC mimetic, IL-15, N-803, an IL-15 derivative, and an autophagy inhibitor. After a period of time, the samples are assayed for surface expression of MHC-I.

Various objects, features, aspects and advantages of the inventive subject matter will become more apparent from the following detailed description of preferred embodiments, along with the accompanying drawing figures in which like numerals represent like components.

### DETAILED DESCRIPTION

The inventive subject matter provides compositions, methods, and kits by which an immune response to a cancer or infectious disease may be enabled or enhanced. An established biomarker of an enabled immune response to exogenous or endogenous non-self immune stimulants is MHC-I expression. By MHC-I it is intended to refer to MHC class I molecules, which are one of two primary classes of major histocompatibility complex (MHC) molecules (the other being MHC class II) and are found on the cell surface of all nucleated cells in the bodies of vertebrates. Their function is the external display on the cell surface of peptide fragments of proteins from within the cell to cytotoxic T cells; this will trigger an immediate response from the immune system against a cell displaying a particular non-self antigen by an MHC class I protein. Because MHC class I molecules present peptides derived from cytosolic proteins, the pathway of MHC class I presentation is often called cytosolic or endogenous pathway. In humans, the HLAs corresponding to MHC class I are HLA-A, HLA-B, and HLA-C.

With respect to NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56bright, CD2, CD7, CD11a, CD28, CD45, and CD54 surface markers and did not display the CD1, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-1a, IL-6, tumor necrosis factor α, interferon α, and interferon γ. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407. U.S. Pat. Nos. 7,618,817, 8,034,332, 8,313,943, 9,150,636, 9,181,322, 10,138,462, and 10,258,649 are herein incorporated by reference in their entirety as are all other extrinsic references.

In another aspect of the inventive subject matter, the genetically engineered NK cell may also be an NK-92 derivative that is modified to express the high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below. U.S. Pat. Nos. 10,738,279, 10,456,420, 10,736,921, 11,000,550, 10,801,013, and 10,774,310

Genetic modification of the NK cells contemplated herein can be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with poly A+ tail) may be used.

For example, where the NK cell is an autologous NK cell or an NK-92 cell it is contemplated that the recombinant nucleic acid will include a segment that encodes a CAR that includes FcεRIγ signaling domain, and preferably also a segment that encodes a cytokine to provide autocrine growth stimulation (e.g., IL-2, IL-2 that is modified with an ER retention sequence, IL-15, or IL-15 that is modified with an ER retention sequence) and/or a segment that encodes a CD16 or high affinity CD16158V. As will be readily appreciated, inclusion of a cytokine that provides autocrine growth stimulation will render the modified recombinant independent of exogenous cytokine addition, which will render large scale production of such cells economically feasible. Likewise, where the modified recombinant also expresses CD16 or a high affinity CD16158V, such cells will have further enhanced ADCC characteristics and with that further improved targeted cytotoxicity.

One should appreciate that the recombinant nucleic acid that encodes that cytokine and/or the CD16 or high affinity CD16158V can be integrated in to the genome of the NK cell, or can be supplied as an extrachromosomal unit (which may be a linear or circular DNA, or a linear RNA, virally delivered or via chemical, mechanical, or electrical transfection). For example, recombinant NK-92 cells expressing IL-2ER and CD16158V are known as haNK cells (Oncotarget 2016 Dec. 27; 7(52): 86359-86373) and can be transfected with a recombinant nucleic acid that includes a segment that encodes a CAR that includes FcεRIγ signaling domain. Once more, such recombinant nucleic acid may comprise further segments that may encode additional immunotherapeutic proteins, such as N-803, TxM-type compounds, IL-8 traps, TGF-β traps, etc. Likewise, NK-92 cells may already be transfected with a cDNA that encodes IL-2 (e.g., NK-92 MI, ATCC CRL-2408). Such cells can then be further transfected with a recombinant nucleic acid that includes a segment that encodes a CAR that includes FcεRIγ signaling domain along with a segment that encodes a CD16 or high affinity CD16158V.

On the other hand, (e.g., autologous, fresh, cultivated, or previously frozen) NK cells or NK-92 cells may also be transfected with a recombinant nucleic acid that includes a segment that encodes a CAR with a FcεRIγ signaling domain, a segment that encodes a cytokine to provide autocrine growth stimulation (e.g., IL-2, IL-2 that is modified with an ER retention sequence, IL-15, or IL-15 that is modified with an ER retention sequence) and a segment that encodes a CD16 (SEQ ID NO:34) or high affinity CD16158V (SEQ ID NO:35, encoded by SEQ ID NO:36) as further disclosed in PCT/US2019/033407, which is incorporated by reference herein in its entirety. Most typically, such recombinant nucleic acid will be arranged as a tricistronic construct. As noted before, such constructed can be an extrachromosomal circular plasmid, a linear DNA (which may be integrated into the genome of the NK cell), or a linear RNA. Such nucleic acids will typically be transfected into the cells in a manner well known in the art (e.g., electroporation, lipofection, ballistic gene transfer, etc.). Similarly, the nucleic acid may be delivered to the cell via a recombinant virus. Therefore, NK cells suitable for use herein include NK-92 cells (which may be transfected with a tricistronic construct encoding a CAR, a CD16 or variant thereof, and a cytokine or variant thereof), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR). U.S. patent application Ser. No. 17/056,385 and U.S. Pat. Nos. 11,077,143, 10,738,279, U.S. patent application Ser. No. 16/969,152 are herein incorporated by reference.

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity.

Consequently, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, the vectors described herein allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5). Alternatively, or additionally, the vector is a plasmid vector. In one embodiment, the vector is a viral vector. As would be understood by one of skill in the art, any suitable vector can be used, and suitable vectors are well-known in the art.

In still other embodiments, the cells are transfected with mRNA encoding the protein of interest (e.g., the CAR). Transfection of mRNA results in transient expression of the protein. In one embodiment, transfection of mRNA into NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK or NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and an FcεRIγ signaling domain.

In preferred embodiments, the cytoplasmic domain of the CAR comprises or consists of a signaling domain of FcεRIγ. Notably, and as described in more detail below, the FcεRIγ signaling domain provide for substantially increased expression levels of the CAR as much as for significantly extended cytotoxicity over time. In some embodiments, the FcεRIγ cytoplasmic domain is the sole signaling domain. However, it should be appreciated that additional elements may also be included, such as other signaling domains (e.g., CD28 signaling domain, CD3ζ signaling domain, 4-1BB signaling domain, etc.). These additional signaling domains may be positioned downstream of the FcεRIγ cytoplasmic domain and/or upstream of the FcεRIγ cytoplasmic domain. In alternative embodiments, the cytoplasmic domain of the CAR may also comprise a signaling domain of CD3 zeta (CD3ζ). In one embodiment, the cytoplasmic domain of the CAR consists of a signaling domain of CD3 zeta.

Therefore, contemplated CARs will include a general structure of a desired antigen binding domain that is coupled to a hinge domain, which is coupled to a transmembrane domain, which is coupled to a signaling domain. Viewed from another perspective, contemplated CARs may have a desired binding domain that is then coupled to a hybrid protein that comprises, consists of, or essentially consists of a hinge domain, which is coupled to a transmembrane domain, which is coupled to a signaling domain.

Most typically, but not necessarily, the extracellular binding domain of the CAR will be a scFv or other natural or synthetic binding portion that specifically binds an antigen of interest. Especially suitable binding portions include small antibody fragments with single, dual, or multiple target specificities, beta barrel domain binders, phage display fusion proteins, etc. Among other suitable extracellular binding domains, preferred domains will specifically bind to a tumor-specific antigen, a tumor associated antigen, or a patient- and tumor-specific antigen. Tumor-specific antigens include, without limitation, NKG2D ligands, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAP, WT-1, PSMA, NY-ESO1, AFP, CEA, CTAGIB, and CD33. Additional non-limiting tumor-associated antigens, and the malignancies associated therewith, can be found in Table 1. Still further tumor-specific antigens are described, by way of non-limiting example, in US2013/0189268; WO 1999024566 A1; U.S. Pat. No. 7,098,008; and WO 2000020460, each of which is incorporated herein by reference in its entirety. Likewise, other preferred domains will specifically bind to a (pathogenic) virus-specific antigen, such as an antigen of an HIV virus (e.g., gp120), an HPV virus, an RSV virus, an influenza virus, an ebolavirus, or an HCV virus.

Consequently, contemplated CARs will target antigens associated with a specific cancer type. For example, targeted cancers include leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In an embodiment, the inventive subject matter comprises a method of treatment for cancer, the method comprising determining the MHC-I expression levels in a tumor. Upon determination that MHC-I levels are low relative to control non-cancerous tissue, the patient is administered a plurality of NK cells, wherein the NK cells comprise at least one of an aNK cell, a haNK cell, a t-haNK cell, or a primary ceNK or a memory-like ceNK (m-ceNK) cell. NK cells may be autologous or allogeneic. The NK cell may be administered intravenously or intratumorally. After a period of time, the patient is administered a plurality of T cells. The T cells may be autologous or allogeneic. The T cell may further be genetically engineered to express a targeting agent, wherein the agent comprises a CAR or a TCR.

A suitable method for determining MHC-I levels in a tumor and normal tissue sample involves measuring cell surface expression of MHC-I. Enzymatic or mechanical dissociation of tissues, whereby viable whole cells are separated and purified from primary tissue, is required for Ab staining and flow cytometric determination of MHC-I surface expression. Alternative methods may include transcriptomic analysis, proteomics, western blotting, and surface plasmon resonance (SPR).

As used herein, a t-haNK cell is an NK cell expressing a genetically engineered CAR. Without being bound by any particular theory, it is understood that the targeting moiety on the CAR is dual purpose. First, the targeting moiety directs the NK cell to the site of expression of the antigen in the patient, thereby facilitating delivery of the NK cell to the tumor tissue. Second, the targeting moiety may be immunogenic, thereby facilitating delivery of antibodies and T cells to the tumor.

Primary NK cells may also be enriched and expanded from whole or cord blood mononuclear cells via standard methods including exposure of the primary NK cells to a CD16 antibody, dexamethasone, and/or IL-15. Stabilized IL-15 may be used, wherein stabilized IL-15 includes IL-15 superagonists such as nogapendenkin alpha imbakicept (Alt-803, N-803, Vesanktiva™), as well as stabilized IL-15/IL-15Ra fusion proteins. U.S. patent application Ser. No. 16/985,728, U.S. patent application Ser. No. 16/505,528, U.S. Pat. No. 11,351,196, U.S. Patent Application No. 63/156,269 U.S. Pat. Nos. 8,163,879, 8,507,222, and 10,537,615 are herein incorporated by reference.

The cytokine enhanced NK (ceNK) cells disclosed herein refers to NK cells in which the cytotoxic activity is enhanced by cytokine stimulation. ceNK cells are prepared by inducing NK cells with a corticosteroid and optionally a cytokine composition comprising IL-15, IL-15:IL-15Rα, or agonist derivatives thereof, such as N-803. The cytokine composition may comprise a fusion protein, wherein the fusion protein comprises IL-15 or an agonist derivatives thereof. Fusion proteins comprising IL-15, wherein the fusion protein has increased stability over IL-15 are preferred. While not limiting the inventive subject matter, it is generally preferred that the corticosteroid is hydroxycortisone and the optional cytokine is N-803.

The Memory-like Cytokine-Enhanced NK Cells (m-ceNK) disclosed herein comprises enriched and expanded NK cells obtained from peripheral blood of donors using the apheresis technique to generate NK cells with a memory-like phenotype. The m-ceNK cells exhibit both high cytotoxicity and increased interferon-gamma production. These m-ceNK cells can be generated from an individual donor for autologous cell therapy or may be generated as an allogeneic product from cord blood. In addition to the enhanced efficacy, m-ceNK cells can be infused easily in an outpatient setting.

As a non-limiting example, the m-ceNK cells may be generated by a step of obtaining a plurality of mononuclear cells and contacting the plurality of mononuclear cells with a corticosteroid and optionally a cytokine. In another step, the plurality of mononuclear cells are incubated in the presence of the corticosteroid and the optional cytokine to enrich the mononuclear cells in NK cells, and the enriched NK cells are then induced with a cytokine composition comprising IL-15, IL-12, and IL-18, or agonist derivatives thereof. The composition may comprise one or more fusion proteins, wherein the fusion proteins comprise at least one of the IL-15, IL-12, and IL-18 cytokines, or agonist derivatives thereof. The cytokine composition may comprise a TxM fusion protein to generate the m-ceNK cells, wherein the TxM fusion protein comprises a protein portion having IL-12 activity, a protein portion having IL-15 activity, and a protein portion having IL-18 activity.

Further description of making the m-ceNK cells and its advantageous properties is described in PCT/US2022/018290, which is incorporated by reference in its entirety. U.S. patent application Ser. No. 17/375,985 and U.S. Pat. No. 11,453,862 provide additional alternative methods of inducing enrichment and expansion of NK cells. Each of the above references are incorporated by reference in its entirety.

### Activated Cytotoxic Cell Therapy

“T cell receptor” or “TCR” refers to a dimeric polypeptide that is typically found on the surface of T cells. Each peptide chain of a TCR generally comprises an extracellular domain comprising a variable region and a constant region, a transmembrane domain, and an intracellular domain. The variable region is the portion of the TCR that interacts with the antigen presented by the MHC. The constant region is the area in each of the two peptides wherein the two peptide chains are covalently linked by a disulfide bond. The intracellular domain generally comprises a CD3z, which comprises one or more immunoreceptor tyrosine-based activation motifs (ITAMs). The ITAM mediates the binding of the variable region to the appropriate intracellular signaling pathways.

The intracellular signaling domain of the CAR may also comprise an FcεRIγ portion. U.S. patent application Ser. No. 17/341,098 is herein incorporated by reference.

A T cell may optionally comprise a modified TCR, which relates to a dimeric polypeptide based on a TCR structure. In particular, the modified TCR comprises two peptide chains, each of which comprise an extracellular domain (comprising a variable region, a constant region, and a connecting peptide), a transmembrane domain, and an intracellular domain. In a specific embodiment, the variable region and constant region are attached via a linker. In another specific embodiment, the connecting peptide is located between the constant region and the transmembrane domain. In a further specific embodiment, the two peptide chains are connected to each other by a disulfide bond between the connecting peptides of each peptide chain. The modified TCR does not interact with endogenous TCR's produced by the T cell. The contents of U.S. Patent Application No. 63/227,195 is herein incorporated by reference.

Once the CAR-based therapeutic (e.g., an antigen binding domain coupled to a CAR scaffold) binds to the antigen expressed by the cancer cell, the cytotoxic cell can trigger destruction of the cancer cell. While it is generally contemplated that all cytotoxic cells are deemed suitable for use herein, especially preferred cytotoxic cells include NK cells, activated NK cells, high affinity NK cells, CD8+ T-cells, and CD4+ T-cells that have been modified to recombinantly express the CAR-based therapeutic, any of which may be of different origins. The cytoxic cell is engineered to express a TCR which recognizes MHC-I presented peptides.

Therapeutic T cells as used herein may be patient-derived (autologous) or donor derived (allogeneic). T cells are typically obtained via leukapheresis, and further separated according to surface marker (CD4, CD8) expression. Purified T cells may be activated by exposure to CD3 and/or CD28 Ab's, or by exposure to antigen presenting cells (APC). Cells may be expanded by exposure to a cytokine cocktail comprising one or more of IL-2, IL-15, and IL-7. In a preferred embodiment, T cells or primary NK cells are expanded on an automated platform and may be transfected by micro-flow through electroporation as described in U.S. Pat. No. 11,377,652, the contents of which are herein incorporated by reference.

T cells may be purified from a tumor, and as such are tumor infiltrating lymphocytes (TIL). TIL may be purified from tumor tissue and expanded ex-vivo. TIL may be reintroduced to the patient as autologous cells or may be administered to a different subject as donor cells. It is anticipated that the stimulation of MHC-I expression by NK cells will lead to enhanced cytotoxic efficacy by TIL.

In an embodiment of the invention, TIL are isolated from a patient tumor by standard techniques. TIL are then exposed to patient tumor tissue, wherein the patient has been treated with NK cells, thereby inducing expression of MHC-I. The NK exposure may be by IV injection, or by intratumoral injection. NK exposure to tumor tissue may be performed ex vivo. TIL exposed to tumor tissue ex vivo are thereby activated and expanded. Expanded TIL comprising CD4 and CD8 cytotoxic T cells are administered to the patient.

T cells may be transfected to express one or more CARs. Transfection techniques include, but are not limited to viral transduction, mRNA transfection, and the Sleeping Beauty transposon system. Subsequent to transfection, CAR T cells may be expanded in a bioreactor until a clinically effective number of cells is obtained.

However, it should be appreciated that in other aspects, the cytotoxic cell may also be a macrophage, a monocyte, a neutrophil cell, a basophile, or eosinophil cell. Therefore, and viewed from a different perspective, the cells contemplated herein may effect cytotoxic action via phagocytosis, pore formation, induction of antibody-dependent cell-mediated cytotoxicity (ADCC), by triggering TNF or fas mediated killing pathways, etc.

Cytotoxic cells may release various types of cytotoxic granules (e.g., granulysin, perforin, granzymes) as part of the cytotoxic anti-tumor process. A variety of assays are available for monitoring cell-mediated cytotoxicity, including flow cytometric assays, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &: 1127-1135).

In one embodiment, a bodily fluid is obtained subsequent to treatment with NK cells, wherein the bodily fluid comprises cellular components, e.g., tumorigenic or cancer cells displaying an antigen to which the CAR-expressing cytotoxic cells described herein bind to, and cytotoxic cells expressing the antigen binding moiety are contacted with the cells. Assays are then performed to detect immune responses, e.g., indicating that an ADCC response or an ADCP response has been triggered by the patient's own immune cells.

Assays for detecting an immune response are known in the art and are described herein. For example, assays for detecting such a response may detect a release of cytotoxic granules (e.g., granulysin, perforin, granzymes), or phagocytosis, or receptor-ligand mediated cytolysis (e.g., as mediated by the Fas/APO pathway). A variety of flow cytometric assays are available for monitoring cell-mediated cytotoxicity, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &: 1127-1135).

In other embodiments, immune stimulatory cytokines are administered to a patient in combination with the cytotoxic cell (expressing a CAR-based therapeutic (e.g., an antigen binding domain coupled to a CAR scaffold) to promote or trigger an immune response. Cytokines include but are not limited to IL2, IL4, IL7, IL11, IL15, IL21, TNF-alpha, IFN-gamma, etc. In some embodiments, cytokines can reactivate exhausted T cells. In other cases, immune competent cells may be engineered to recombinantly express one or more cytokines.

Other techniques to treat cancer include surgery, radiation therapy, chemotherapy, immunosuppressive reagents (e.g., azathioprine, cyclosporin, methotrexate, mycophenolate, etc.), immunotherapy, targeted therapy, hormone therapy, stem cell transplant, or other precision methods. Any of these techniques may be combined with embodiments of the present invention to treat cancer.

It is understood that present invention embodiments may be administered to a patient using appropriate formulations, indications, and dosing regimens suitable by government regulatory authorities such as the Food and Drug Administration (FDA) in the United States.

In some embodiments, a cytotoxic cell expressing a TCR, a modified TCR, or a CAR-based therapeutic (e.g., an antigen binding domain coupled to a CAR scaffold) is administered to a patient as a pharmaceutical composition. In another embodiment, a method of treating cancer by administration of the cytotoxic cell to a subject is contemplated. In still another embodiment, a method inhibiting the proliferation or reducing the proliferation of a cell that is expressing the corresponding antigen (to which the antigen binding region binds to) on the surface of its cell by administration of the cytotoxic cell to a subject is contemplated.

In an embodiment, the patient may be lymphodepleted, whereby endogenous lymphocyte numbers are reduced, thereby increasing the availability of essential endogenous cytokines and promoting infused T cell survival.

In some embodiments, the cytotoxic cell expressing a TCR, a modified TCR, or a CAR-based therapeutic (e.g., an antigen binding domain coupled to a CAR scaffold) reduces the amount (e.g., number of cells, size of mass, etc.) by at least 25%, at least 50%, at least 75%, at least 90%, at least 95%, or at least 99% in a subject with cancer associated with expression of the corresponding antigen on the surface of the cells relative to a negative control.

Examples of cancer that are treatable by the cytotoxic cells contemplated herein include any cancer expressing or overexpressing a cancer-associated antigen on its cell surface. Examples of cancer that can be treated with a cytotoxic cell expressing a TCR, a modified TCR, or a CAR-based therapeutic (e.g., an antigen binding domain coupled to a CAR scaffold) include but are not limited to breast cancer, colon cancer, leukemia, lung cancer, melanoma, neuroblastoma, pancreatic cancer, pediatric intracranial ependymoma, and prostate cancer.

Pharmaceutical compositions may comprise cytotoxic cells comprising an antigen binding domain coupled to or linked to a CAR scaffold, as described herein, in combination with one or more pharmaceutically or physiologically acceptable carriers, diluents or excipients. Additionally, pharmaceutical compositions may comprise one or more adjuvants (e.g., aluminum hydroxide), antioxidants, bacteriostats, buffers, carbohydrates, chelating agents such as EDTA or glutathione; coloring, flavoring and/or aromatic substances, emulsifiers, excipients, lubricants, pH buffering agents, preservatives, salts for influencing osmotic pressure, polypeptides (e.g., glycine), proteins, solubilizers, stabilizers, wetting agents, etc., which do not deleteriously react with the active compounds (e.g., antigen binding domain coupled to a CAR scaffold, etc.) or otherwise interfere with their activity. Buffers include but are not limited to neutral buffered saline, phosphate buffered saline, etc. Carbohydrates include but are not limited to dextrans, glucose, mannose, mannitol, sucrose, etc.

Pharmaceutical compositions may be formulated for a particular mode of administration. Modes of administration may include but are not limited to: intraarticular, intradermal, intranasal, intraperitoneal, intrathecally, intratumoral, intravenous, intraventricularly, subcutaneous, transdermal, transmucosal or topical routes.

In preferred embodiments, the cytotoxic cells are administered by intravenous infusion. Such formulations may be prepared according to standard techniques known by one of ordinary skill in the art. For example, a composition that is to be administered intravenously may have one or more ingredients (e.g., a diluent, a suspension buffer, saline or dextrose/water, other components such as cytokines, etc.) prior to infusion in the patient.

Many such techniques for formulating and administering pharmaceutical compositions are known in the art, e.g., U.S. Patent Application Publication No. 2014/0242025, and all such references are incorporated by reference herein in their entirety.

In some embodiments, the cytotoxic cells proliferate in vivo, thereby persisting in the patient for months or even years after administration to provide a sustained mechanism for inhibiting tumor growth or recurrence. In some aspects, the cytotoxic cells persist at least for three months, six months, nine months, twelve months, fifteen months, eighteen months, two years, three years, four years, or five years after administration of the cytotoxic cells to the patient.

Cytotoxic cells may be obtained from any of a variety of sources, (e.g., isolated from a human, from commercially available cytotoxic cells, from a cell repository, etc.). Procedures for ex vivo expansion of NK cells, T cells or other types of cytotoxic cells are known in the art (e.g., Smith et al., Clinical & Translational Immunology (2015) 4: e31). The examples presented herein are not intended to be limited to any particular method of ex vivo expansion of cytotoxic cells.

Pharmaceutical compositions comprising cytotoxic cells, as described herein, may be administered at a dosage of 104 to 109 cells/kg body weight, of 105 to 106 cells/kg body weight, or any integer values within these ranges. Cytotoxic cell compositions may be administered one time or serially (over the course of days or weeks or months) at these dosages. Infusion techniques for cytotoxic cells, such as T cells, are known in the art (see, e.g., Rosenberg et al., New Eng. J. of Med. 319:1676, 1988).

In other embodiments, the pharmaceutical compositions are administered in a therapeutically effective amount, which is the amount effective for treating the specific indication. Administration may occur as a one-time dose or based on an interval. As used herein, “interval” indicates that the therapeutically effective amount is administered periodically (as distinguished from a one-time dose). The administration interval for a single individual need not occur at a fixed interval, but can vary over time. The term, “in combination with” or “co-administered” indicates that a composition can be administered shortly before, at or about the same time, or shortly after another composition.

### EXAMPLES

### Example 1

aNK, haNK, HER2 t-haNK, and CD19 t-haNK cells are administered to immune deficient mice bearing HER2 or CD19 expressing tumor xenografts. Cells are administered as a whole cell preparation or a lysate. Whole cells and lysates are administered either intratumorally or intravenously. Tumors are then monitored for expression of MHC-I, MHC-II, B2M, and CIITA. MHC-I and MHC-II are monitored for cell surface expression by flow cytometry. Vehicle treated mice are used as a control.

### Example 2

A cancer patient biopsy comprising cancer cells is assayed for expression of MHC-I. The assay includes expression of MHC-I in non-cancerous tissue, preferably from the same tissue source as the biopsy. The expression of MHC-I in the cancerous cells should be at least statistically significantly (p>0.05) lower in three cancerous samples vs. 3 control samples. Preferably, the expression of MHC-I in the cancer cells should be 80%, 60%, 40%, 20%, or <10% of the expression of MHC-I in the normal cells. Preferably, the expression of MHC-I in the cancer cells is <10% of that in control cells.

### Example 3

A cancer patient having a tumor that has been determined to have low MHC-I expression is treated with an NK cell or an NK cell lysate. The treatment is by intratumoral injection. Within 1-7 days, the tumor is re-evaluated for MHC-I expression. Increased expression indicates the patient is now an appropriate candidate for T cell treatment. T cells are derived from 1) the tumor as TIL, 2) the lymph nodes, or 3) peripheral blood. The T cells may be combined. They are grown to a clinically efficacious amount in vitro. The T cells are administered to the patient by intravenous or intratumoral injection.

The above discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the inventive subject matter and does not pose a limitation on the scope of the inventive subject matter otherwise claimed. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of treating a patient having a MHC-I negative tumor tissue, the method comprising sequential administration of an effective amount of a first composition comprising an NK cell and IL-15 or an agonist derivative thereof and then an effective amount of a second composition comprising a T-cell and IL-15 or an agonist derivative thereof, optionally wherein the agonist derivative of IL-15 is N-803 (nogapendenkin alpha imbakicept).

2. The method of claim 1, wherein the NK cell is an NK-92 cell or genetically modified derivative thereof, an activated cytokine-enriched NK (ceNK) cell, or a memory-like ceNK (m-ceNK) cell.

3. The method of claim 2, wherein the NK cell is induced or genetically engineered to overexpress interferon gamma (IFNγ).

4. The method of claim 3, wherein the NK cell comprises at least one of a chimeric antigen receptor (CAR) and an Fc receptor.

5. The method of claim 4, wherein the CAR is targeted to the MHC-I-negative tumor tissue or wherein the CAR is targeted to a tumor associated antigen or a tumor specific antigen.

6. (canceled)

7. The method of claim 4, wherein the NK cell releases IFNγ upon CAR-mediated engagement of the NK cell and the tumor tissue.

8. The method of claim 2, further comprising administering an antibody concurrently with the NK cell, wherein the antibody is targeted to the MHC-I-negative tumor tissue, and optionally wherein the antibody is targeted to a tumor associated antigen or a tumor specific antigen, and further optionally wherein the NK cell releases IFNγ upon Ab-mediated engagement of the NK cell and the tumor tissue.

9. (canceled)

10. (canceled)

11. The method of claim 1, wherein administering the NK cell increases expression of MHC-I in the tumor.

12. The method of claim 1, further comprising administering concurrently with the NK cell at least one therapeutic agent selected from the group comprising Gemcitabine, IFNγ, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, a SMAC mimetic, and an autophagy inhibitor.

13. The method of claim 1, further comprising administering IFNγ concurrently with the NK cell.

14. (canceled)

15. The method of claim 1, wherein the T cells are genetically engineered to express at least one of a CAR and a T cell receptor (TCR), and optionally wherein the at least one CAR and TCR are specific for a tumor associated antigen, a tumor specific antigen, or a neoepitope.

16. (canceled)

17. A pharmaceutical composition for use in for patients having MHC-I negative tumor tissues, the composition comprising 1) an effective amount of an NK cell and IL-15 or an agonist derivative thereof, and 2) an effective amount of a T-cell and IL-15 or an agonist derivative thereof, wherein the NK cells and the T cells are sequentially administered.

18. The composition of claim 17, wherein the NK cell is an NK-92 cell or an activated cytokine-enriched NK (ceNK) cell or a memory-like ceNK (m-ceNK) cell, optionally wherein the NK cell is induced or genetically engineered to overexpress interferon gamma (IFNγ), and further optionally wherein the NK cell comprises at least one of a chimeric antigen receptor (CAR) and an Fc receptor.

19. (canceled)

20. (canceled)

21. The composition of claim 18, wherein the CAR is specific for the MHC-I-negative tumor tissue, and optionally wherein the CAR is specific for a tumor associated antigen or a tumor specific antigen.

22. (canceled)

23. The composition of claim 17, further comprising an antibody to be administered concurrently with the NK cell, wherein the antibody is specific for the MHC-I-negative tumor tissue, optionally wherein the antibody is specific for a tumor associated antigen or a tumor specific antigen.

24. (canceled)

25. (canceled)

26. The composition of claim 17, further comprising at least one therapeutic agent selected from the group comprising Gemcitabine, IFNγ, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, a thymidylate synthetase inhibitor, Dexamethasone, and an autophagy inhibitor, wherein the at least one therapeutic agent is administered concurrently with the NK cell.

27. The composition of claim 17, further comprising IFNγ, formulated for concurrent administration with the NK cell.

28. The composition of claim 17, wherein the T cells are genetically engineered to express at least one of a CAR and a T cell receptor (TCR), and optionally wherein the at least one CAR and TCR are specific for a tumor associated antigen, a tumor specific antigen, or a neoepitope.

29. (canceled)

30. The composition of claim 17, wherein the IL-15 derivative is N-803 (nogapendenkin alpha imbakicept).

31. A method of determining MHC-I inducibility of a tumor, the method comprising:
a. quantifying ex vivo MHC-1 expression in a tumor tissue of a patient;
b. treating the tumor tissue ex vivo with at least one agent selected from a group comprising Gemcitabine, IFNγ, HDAC inhibitors, 5-aza-2′-deoxycytidine, a DNA Methyltransferase inhibitor, Hydralazine, Valproic acid, a micro RNA (miRNA), or an inhibitor of ALK, RET, or MAPK kinases, a glycosyltransferase inhibitor, or a thymidylate synthetase inhibitor;
c. quantifying MHC-I expression in the ex vivo treated tumor tissue;
d. identifying at least one agent wherein the expression level of MHC-I is significantly increased by the treatment.
